(19) United States
(12) Patent Application Publication
(10) Pub. No.: US 2012/0265244 A1
(43) Pub. Date: Oct. 18, 2012

(54) PACIFIER WITH A STERILIZING CASE THEREFOR

(76) Inventors: VINCENT VALDERRAMA, Stamford, CT (US); Diana Sierra, North Bergen, NJ (US)

(21) Appl. No.: 13/447,933

(22) Filed: Apr. 16, 2012

Related U.S. Application Data
(60) Provisional application No. 61/475,771, filed on Apr. 15, 2011.

(51) Int. Cl. A61J 17/00; A61L 2/18

(52) U.S. Cl. 606/234; 422/300

(57) ABSTRACT

A pacifier assembly has a pacifier having a shield with a convex surface and a concave surface. The pacifier has a back portion extending from the convex surface; and a teat extending from the concave surface. The convex surface has a plurality of undulations and a plurality of apertures extending through the shield. The pacifier assembly further includes a case. A pacifier assembly provides for a pacifier having a shield with a convex surface and a concave surface, a back portion extending from the convex surface and a teat extending from the concave surface. The pacifier assembly further provides for a case having a base and a cover. The base includes at least two compartments. One of the at least two compartments includes chamber for receiving fluid and the other of the at least two compartments is sized to receive the pacifier.

### CROSS-REFERENCE TO RELATED APPLICATION

This application claims the benefit of U.S. Provisional Application No. 61/475,771 filed on Apr. 15, 2011, which is incorporated by reference herein.

### BACKGROUND OF THE DISCLOSURE

1. Field of the Disclosure

The present disclosure relates generally to a pacifier for a child and a sterilizer case that is used to sterilize the pacifier. More particularly, the present disclosure relates to a pacifier that protects the face of the infant and a sterilizer case for the pacifier that holds an amount of water to sterilize such pacifier or pacifiers while in the case.

2. Description of Related Art

There exist many different shapes of pacifiers for children. While many parents use pacifiers, they do pose some drawbacks for children. In particular, pacifier use for an extended period of time results in salivation between the child's face and the portion of the pacifier that touches the child's face. Extended and repeated exposure of the sensitive skin around the infant's mouth and chin, in particular, to saliva can cause irritation and potentially a rash.

Additionally, parents typically use pacifiers to sooth or relax the child or to help the child sleep. However, current pacifier designs can interfere with a child's sleep. Pacifier designs often feature a movable element, such as a handle or a ring. These movable elements can be a source of noise due to contact between the pacifier shield and the movable element. The movable element can often be interfered with during the infant's unintentional movement while sleeping and thereby dislodge the pacifier from the child's mouth, causing the infant to wake.

Further, such pacifiers must frequently be sterilized to eliminate any bacteria that has formed on the surfaces of such pacifiers. Sterilization is generally accomplished by immersing the pacifier in boiling water. Boiling water can be inconvenient, time consuming and potentially dangerous. Many current microwavable pacifier cases contain fill lines that are either unclear or become unclear after extended use. Fill lines are often difficult to read or comprehend. Further, once the sterilization cycle is complete, the remaining excess water causes a burn hazard upon removing the pacifier from the microwave oven because there is no way to easily or safely remove excess water.

Accordingly, there exists a need for a pacifier that is structured to minimize the possibility of irritation or a rash on a child's face. There also is a need to prevent movement of parts, in particular, a handle or ring on the pacifier to reduce the likelihood of waking the child during sleep. Further, there is a need for a simple and safe way to properly sterilize pacifiers and to prevent the use of excessive water during the sterilizing process.

### SUMMARY OF THE DISCLOSURE

The present disclosure provides a pacifier that is structured to minimize contact of saliva with the face of the child.

The present disclosure also provides a pacifier case that permits effective and safe sterilization of the pacifier by identifying the proper amount of water needed for sterilizing and permitting safe disposal of any excess water after sterilization.

A pacifier assembly provides for a pacifier having a shield with a convex surface and a concave surface. The pacifier has a back portion extending from the convex surface; and a teat extending from the concave surface. The convex surface has a plurality of undulations and a plurality of apertures extending through the shield. The pacifier assembly further includes a case.

A pacifier assembly provides for a pacifier having a shield with a convex surface and a concave surface; a back portion extending from the convex surface; and a teat extending from the concave surface. The pacifier assembly further provides for a case having a base and a cover, in which the base includes at least two compartments. One of the at least two compartments includes chamber for receiving fluid and the other of the at least two compartments is sized to receive the pacifier.

A case for sterilizing having a base and a cover, the cover pivots relative to the base to be have an opened configuration or a closed configuration. The base includes at least two compartments. One of the at least two compartments includes a chamber for receiving fluid and the other of the at least two compartments is sized to receive a pacifier.

### BRIEF DESCRIPTION OF THE DRAWINGS

FIG. 1 illustrates a front perspective view of a pacifier according to a first embodiment of the present disclosure.

FIG. 2 illustrates a front view of the pacifier of FIG. 1.

FIG. 3 illustrates a cross section view of the pacifier of FIG. 1, taken along line A-A of FIG. 2.

FIG. 4 illustrates a detailed view of the inner surface of the pacifier of FIG. 1.

FIG. 5 illustrates a rear view of the pacifier of FIG. 1.

FIG. 6 illustrates a side view of the pacifier of FIG. 1.

FIG. 7 illustrates a cross-section view taken along line B-B of FIG. 6.

FIG. 8 illustrates a perspective view of a pacifier according to a second embodiment of the present disclosure.

FIG. 9 illustrates a front view of the pacifier according to FIG. 8.

FIG. 10 illustrates a perspective view of a pacifier sterilization case according to the present disclosure.

FIG. 11 illustrates a front view of the pacifier sterilization case according to the present disclosure.

FIG. 12 is a rendition of two pacifiers according to the embodiment of FIG. 1, disposed in the pacifier sterilizing case according of FIG. 10, of the present disclosure.

FIG. 13 is a rendition of two pacifiers according to the embodiment of FIG. 8; disposed in the pacifier sterilizing case of FIG. 10, of the present disclosure.

### DETAILED DESCRIPTION OF THE PREFERRED EMBODIMENTS

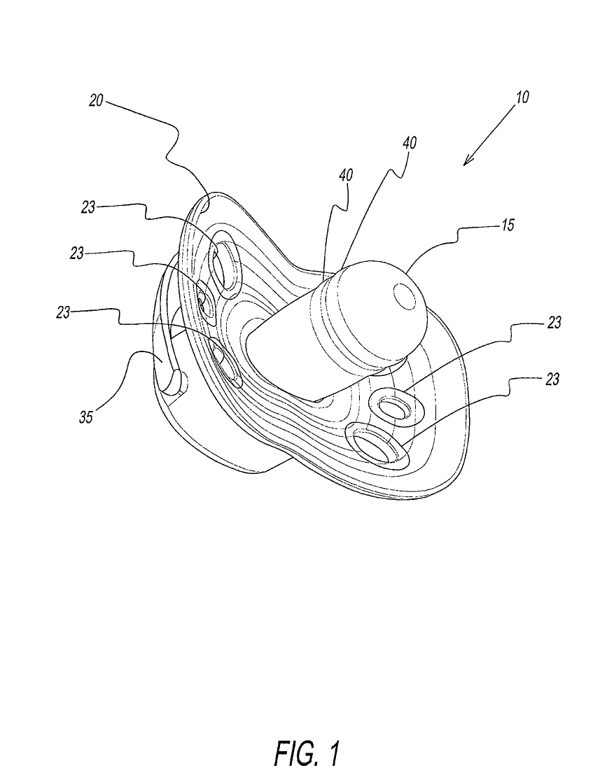

Referring to the drawings and, in particular, to FIG. 1, a pacifier, according the first embodiment of the present disclosure, is generally represented by reference numeral 10. Pacifier 10 includes shield 20 having a teat 15 extending from one side thereof and a back portion 30 extending from an opposite side thereof. Pacifier 10 has a back portion 30 that extends from shield 20 opposite teat 15. Teat 15 is securely fixed between shield and back portion 30 to prevent any possibility of disengagement of teat 15 from pacifier 10. Back portion 30 has a ring or handle 35 that a user can hold to remove pacifier 10 from child's mouth. Handle 35 is rotatable secured to back portion 30 for free movement. Teat 15 has ribs 40 to aid in relaxation or soothing child during use. Shield 20 has several through holes or apertures 23 that prevent pacifier 10 from becoming stuck on an infant or child's (‘Infant“) face and prevent the infant's airway from being obstructed should the pacifier become lodged in the infant's mouth.

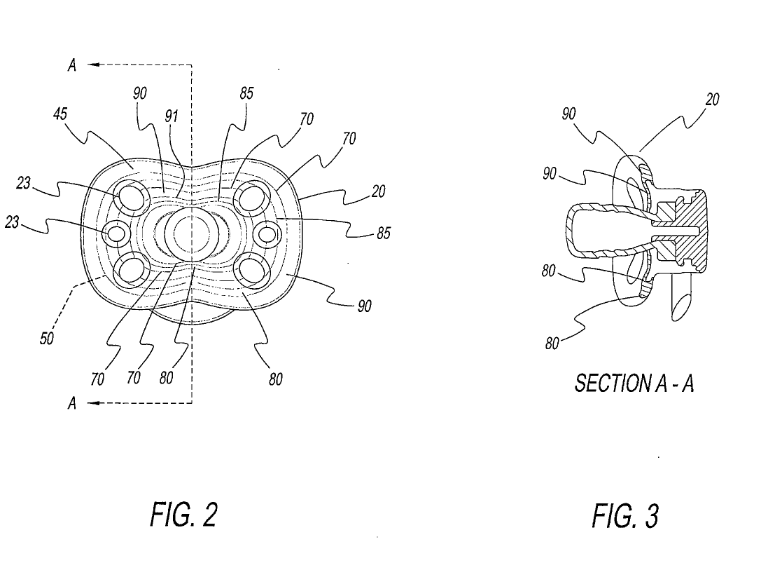

Referring to FIGS. 2 and 3, shield 20 is a gradually curved element that has a generally convex inner surface 45 that is contoured to address the area around the mouth of the infant. Shield 20 has a generally convex outer surface 50 that is away from the face and mouth of the infant during use. Inner surface 45 has a center 55 that intersects longitudinal axis 60 of teat 15. Inner surface 45 is an undulating surface having rises 80 and channels 90. Each rise 80 has a crest 85 and lower edges 70. Rises 80 are immediately adjacent to channels 90. Channels 90 each have lowest point or trough 91.

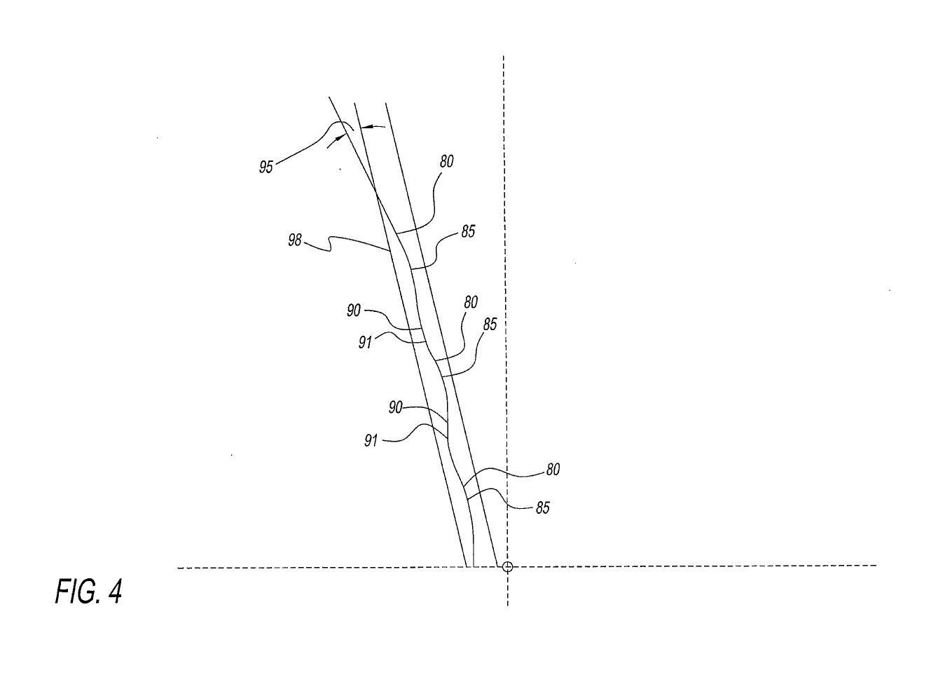

Referring to one embodiment of the present disclosure shown in FIG. 4, each rise 80, in particular crests 85, has a height, ranging from about 0.25 mm to about 1 mm, relative to each trough 91 of each channels 90. Each rise 80 is between an adjacent pair of channels 90 to provide alternating rises and channels. In another embodiment of the present disclosure, crests 85 have a height ranging from about 0.3 mm to about 0.6 mm when measured from height of crests 85 to and adjacent trough 91. In another embodiment, rises 80 have a height of about 0.4 mm. Rises 80 have an angle 95 of about 1 degree to 45 degrees from line 98. In one embodiment rises 80 have an angle 95 of about 20 degrees to about 30 degrees from line 98. In another embodiment, rises 80 have an angle 95 of about 25 degrees from line 98. Line 98 is parallel to or tangential to troughs.

The purpose of alternating rises 80 and channels 90 is to prevent undulating surface 45 from being entirely in contact with the skin of the infant during use.

Alternating rises 80 and channels 90 ensure that surface 45 is only partially in contact with the skin of the infant. In particular, rises 80 are only in contact with the skin of the infant. Channels 90 direct saliva away from the face of the infant and away from rises 80. By minimizing contact of inner surface 45 with face, the amount of saliva in contact with the infant's face is minimized and the possibility of irritation or rash from saliva interacting with face is minimized.

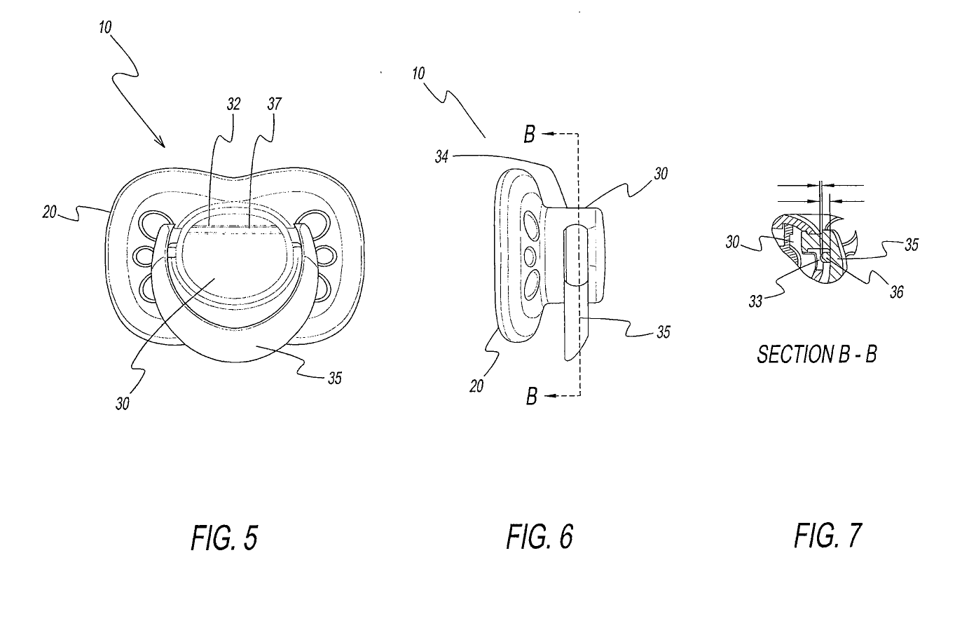

Referring to FIGS. 5 through 7, pacifier 10 has a handle 35 with a post 37 that enables free rotation of handle 35 in throughhole 32 of back portion 30. Handle 35 has a molded male member 36 in the form of an extension 38. A female detent 33 is molded into wall 34 of back portion 30. When male member 36 of handle 35 is rotated past female detent 33 of back portion 30, male member 36 becomes seated in female detent 33 to prevent relative movement between handle 35 and back portion 30. By preventing relative movement between handle 35 and back portion 30, handle 35 will not move when the infant is sleeping. Motion of handle 35 relative to back portion 30 and sleeping surface can dislodge pacifier from mouth of the infant and thereby wake the infant. By preventing relative movement between handle and back portion 30, any noise created from contact between handle 35 and back portion 30 is also eliminated.

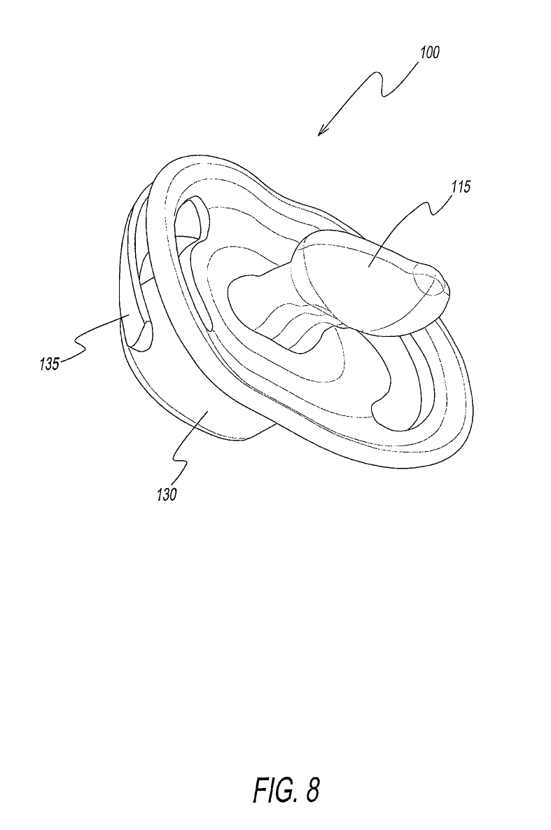

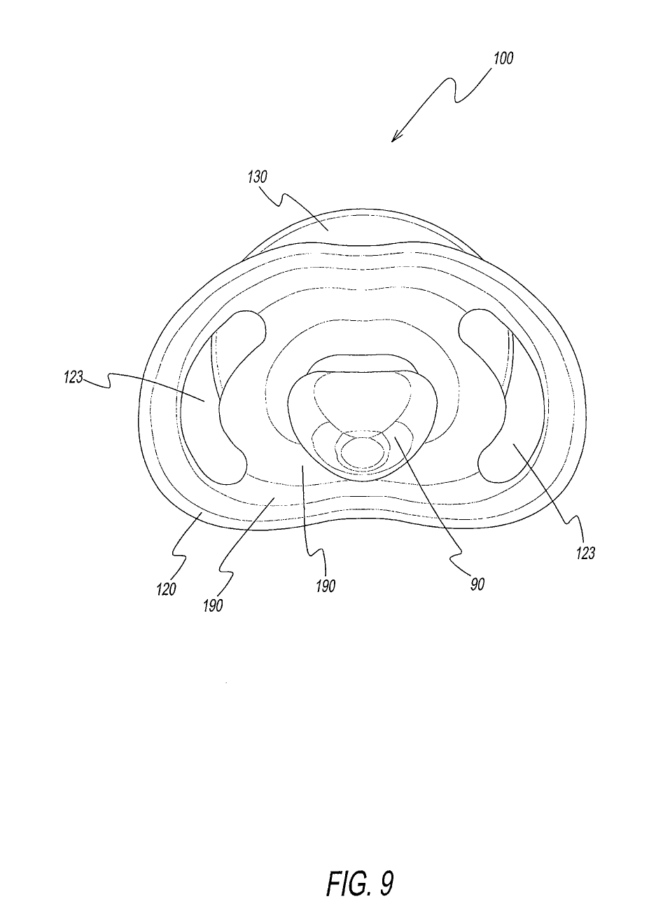

FIGS. 8 and 9 show a second embodiment of pacifier, generally referred to by reference numeral 100. Pacifier 100 has a teat 115 and a shield 120. Pacifier has a handle 135 and a back portion 130. Shield 120 has slots 123 that prevent pacifier 100 from becoming stuck on an infant's face and prevent the infant's airway from being obstructed should pacifier 100 become lodged in the infant's mouth. Pacifier 100 has an inner surface 145 that has rises 180 and channels 190. Rises 180 and channels 190 function to prevent the entire inner surface 145 from lying against face of child and causing irritation or a rash. Further, channels 190 direct saliva away from face of child. Pacifier 100 has a male member on handle 135 and female detent on back portion 130 to prevent relative movement between handle 135 and back portion 130, which performs an identical function as male member 36 and female detent 33 of the embodiment of FIG. 1 through 7.

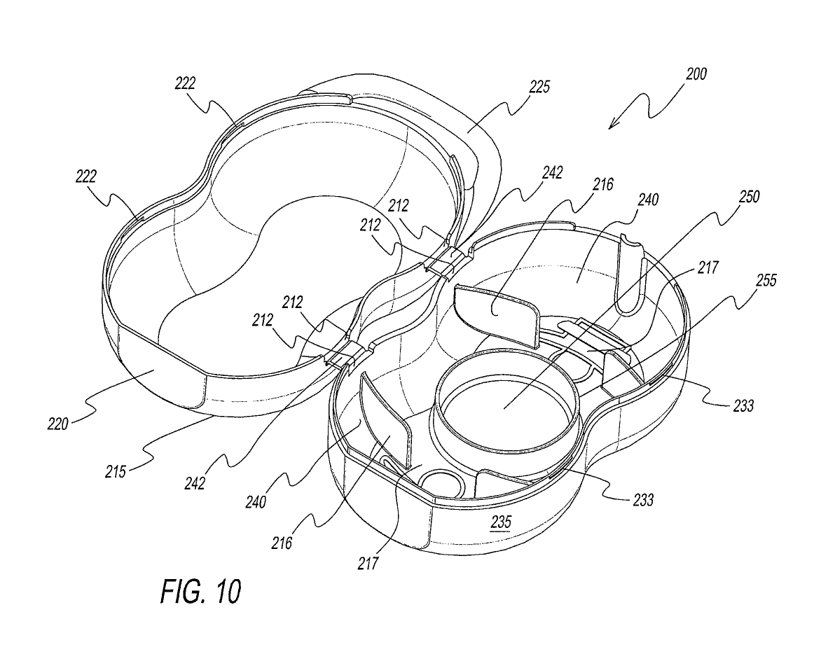

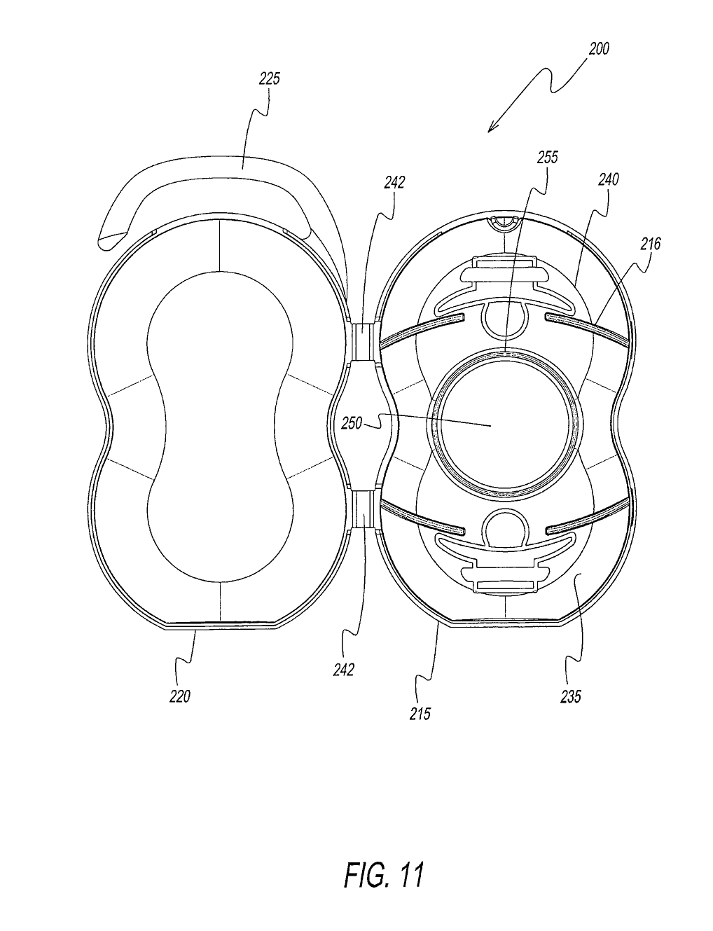

Referring to FIGS. 10 and 11, a sterilizing case is shown, and generally referred to by reference numeral 200. Sterilizing case 200 may be used together with pacifier 10 and pacifier 100, of the present disclosure. Sterilizing case 200 has a water holding chamber 250 that is sized to sterilize the contents of sterilizing case 200. Sterilizing case 200 has a housing 215 with a cover 220 and a base 235 that are pivotally connected by hinges 242. Cover 220 has a handle 225 to permit carrying or holding of sterilizing case 200. Cover 220 has recesses 222, and base 235 has protrusions 233 that fit into recesses 222 to permit sterilizing case 200 to be securely closed during use. Sterilizing case 200 is made from a plastic that remains stable after repeated uses in a microwave over during sterilization.

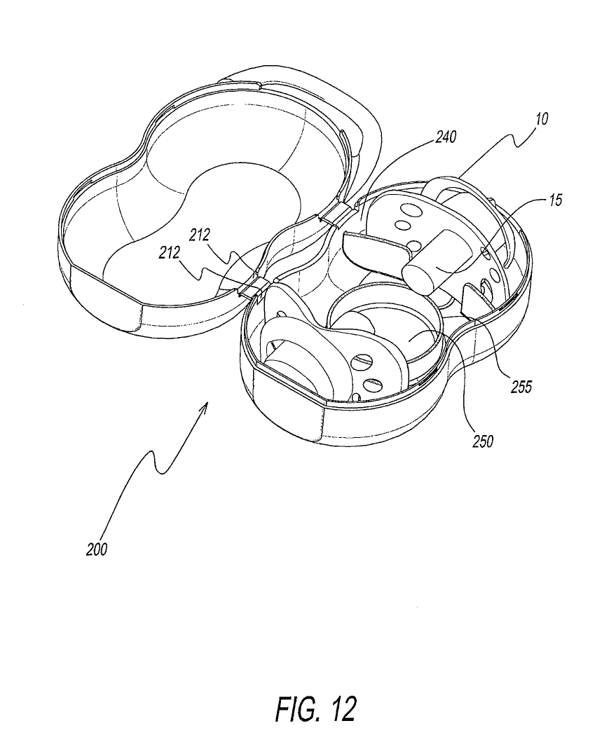

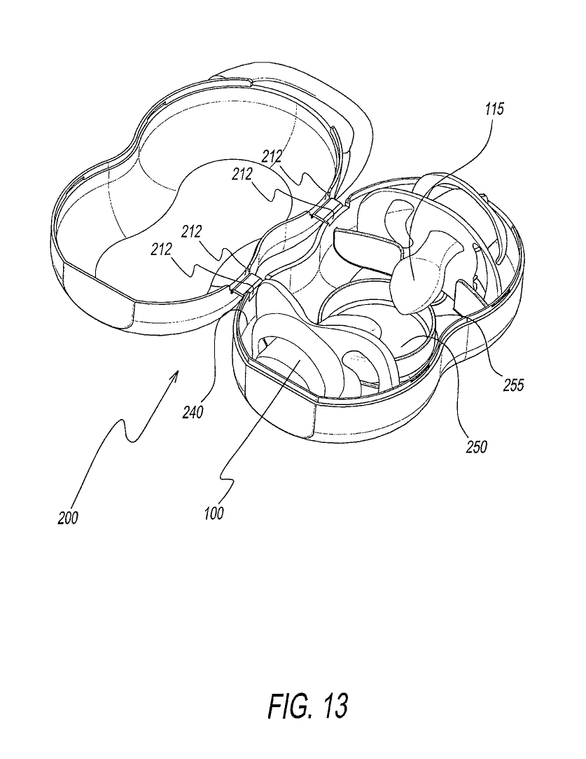

Base 235 has two compartments 240 defined by a partial wall or ribs 216 and housing 215, which compartments are sized to receive two pacifiers, as shown in FIGS. 12 and 13, respectively. Partial wall or ribs 216 have an opening 217 and are designed and configured such that when a pacifier is placed in compartment 240, teat 15,115 extends through opening 217 unimpeded and cannot touch any part of base 235. Accordingly, teat 15,115 is completely surrounded by steam during the sterilization process. Sterilizing case 200 has a third compartment 241 having a chamber 250 with a rim 255. When pacifier is placed in one of the two compartments 240, teat 15, 115 extend into third compartment 241 in a cantilevered fashion. Chamber 250 is sized to hold the precise amount of water that when boiled will sterilize contents of sterilizing case, in particular, two pacifiers. Chamber 250 is sized to hold approximately 8.5 milliliters of water that can be transformed and boiled to create steam. Sterilizing case 200 has vents 212 that permit steam to escape as water boils during sterilization. Significantly, chamber 250 does not contain any lines or markings that reference any water level. In contrast, chamber 250 is filled to rim 255 to provide the correct amount of water that needs to be in sterilizing case 200 to achieve proper sterilization without having to use any markings that may be difficult to read or interpret. Chamber 250 prevents the user from using excessive water, which could present a burning hazard during removal of sterilizing case from microwave oven. Further, vents 212 permit steam to escape from housing 215 so that sterilization case does open and present a safety hazard or defeat the purpose of the sterilizing process.

Case 250 is made from a material that is able to withstand temperatures greater than 121° C. Case 250 may be made from material including acetal, polyimide, polycarbonate, polymethyl methacrylate, polypropylene, polyphenylene sulfide, polystyrene, polysulphone, or poly vinyl chloride, and any combinations thereof.

The present disclosure has been described with particular reference to the preferred embodiments. It should be understood that the foregoing descriptions and examples are only illustrative of the present disclosure. Various alternatives and modifications thereof can be devised by those skilled in the art without departing from the spirit and scope of the present disclosure. Accordingly, the present disclosure is intended to embrace all such alternatives, modifications, and variations that fall within the scope of the appended claims.

We claim:

1. A pacifier assembly comprising:
a pacifier having a shield with a convex surface and a concave surface;
a back portion extending from the convex surface; and a teat extending from the concave surface;
a plurality of undulations on the convex surface; and
a plurality of apertures extending through said shield.

2. The pacifier assembly according to claim 1, wherein said back portion comprises a handle that is able to rotate relative to said back portion and have a fixed relationship relative to said back portion.

3. The pacifier assembly according to claim 2, wherein said back portion further comprises a detent and said handle comprises a male extension that can be seated in said detent to prevent movement of said handle relative to said back portion.

4. The pacifier assembly according to claim 1, wherein said plurality of apertures comprise a plurality of slots or a plurality of through holes that extend through said shield.

5. The pacifier assembly according to claim 1, wherein said plurality of undulations comprise alternating of rises and channels.

6. The pacifier assembly according to claim 4, wherein said rises each comprise a crest and said channels each comprises a trough, and the vertical distance between adjacent crests and troughs ranges from 0.25 mm to 1.00 mm.

7. The pacifier assembly according to claim 6, wherein said rises have an angle ranging from 1° to 45° relative to a line that is tangential to adjacent troughs.

8. The pacifier assembly further comprising a case having a base and a cover connected to the base.

9. The pacifier assembly according to claim 8, where said base comprises a chamber and at least one compartment that is sized to hold the pacifier.

10. The pacifier assembly according to claim 9, wherein said at least one compartment comprises a partial wall that has an opening through which said teat extends without contacting said base.

11. The pacifier assembly according to claim 9, wherein said chamber holds approximately 8.5 milliliters of liquid that can generate steam to sterilize contents of said case.

12. The pacifier assembly according to claim 8, wherein said case comprises vents to release steam.

13. The pacifier assembly of claim 8, wherein said case is made from a material selected from the group consisting of acetal, polyamide, polycarbonate, polymethyl methacrylate, polypropylene, polyphenylene sulfide, polystyrene, polysulphone, or poly vinyl chloride, and any combinations thereof.

14. A pacifier assembly comprising:
a pacifier having a shield with a convex surface and a concave surface;
a back portion extending from the convex surface; and a teat extending from the concave surface; and
a case having a base and a cover, wherein said base comprises at least two compartments, and wherein one of said at least two compartments comprises as chamber for receiving fluid and the other of the at least two compartments is sized to receive said pacifier.

15. The pacifier assembly according to claim 14, wherein said convex surface comprises a plurality of undulations of adjacent of rises and channels, wherein a rises is between two channels.

16. The pacifier assembly according to claim 15, further comprising a distance between a height of one of said plurality of rises and a depth of an adjacent channel ranges from 0.25 mm to 1.00 mm.

17. The pacifier assembly according to claim 14, wherein said back portion further comprises a detent and a handle that is able to rotate relative to said back portion.

18. The pacifier assembly according to claim 17, wherein said handle comprises a male extension that can be seated in said detent to prevent movement of said handle relative to said back portion.

19. The pacifier assembly according to claim 14, wherein said pacifier comprises a plurality of apertures that extend from said convex surface to said concave surface.

20. The pacifier assembly according to claim 19, wherein said plurality of apertures comprise a plurality of slots or a plurality of holes.

21. The pacifier assembly according to claim 14, wherein said at least two compartments are separated by a partial wall that has an opening through which said teat extends is able to extend in a cantilevered fashion without contacting said base.

22. The pacifier assembly according to claim 14, wherein said at least two compartments comprise three compartments, and wherein said case is able to hold two pacifiers.

23. A case for sterilizing comprising:
a case having a base and a cover,
wherein said base comprises at least two compartments, and wherein one of said at least two compartments comprises as chamber for receiving fluid and the other of the at least two compartments is sized to receive a pacifier.

24. The case according to claim 23, wherein said at least two compartments are separated by a partial wall that has an opening through which a teat of a pacifier can extend without contacting said base.

25. The case according to claim 23, wherein said at least two compartments comprise three compartments, and wherein said case is able to hold two pacifiers.

26. The case according to claim 23, wherein said chamber holds approximately 8.5 milliliters of liquid that can generate steam to sterilize contents of said case.

27. The case according to claim 23, wherein said case comprises vents to release steam.

28. The case according to claim 23, wherein said cover pivots relative to said base to provide an opened configuration or a closed configuration.

29. The case of claim 23, wherein said case is made from a material selected from the group consisting of acetal, polyamide, polycarbonate, polymethyl methacrylate, polypropylene, polyphenylene sulfide, ploystyrene, polysulphone, or poly vinyl chloride, and any combinations thereof.
